(19) United States
(12) Patent Application Publication
(10) Pub. No.: US 2011/0064780 A1
(43) Pub. Date: Mar. 17, 2011

(54) POLYMERIC PHEROMONE FORMULATION AND METHOD OF USE TO CALM STRESS-RELATED BEHAVIOR IN MAMMALS OVER AN EXTENDED PERIOD OF TIME

(76) Inventor: GERALD L. MARSHALL, Athens, TX (US)

(21) Appl. No.: 12/877,911

(22) Filed: Sep. 8, 2010

Related U.S. Application Data
(63) Continuation-in-part of application No. 12/430,075, filed on Apr. 25, 2009.
(60) Provisional application No. 61/125,977, filed on Apr. 30, 2008.

(51) Int. Cl. A61K 9/00; A61K 31/575; A61P 43/00

(52) U.S. Cl. 424/402; 514/171

(57) ABSTRACT

The present invention is directed to a polymeric formulation for controlling animal behavior comprising a low melting point polymer or copolymer, an unsaturated long chain fatty alcohol, and an animal-appeasing pheromone to produce a solid slow release generator of the pheromone. The system is useful for making animal collars and the like.

### RELATED APPLICATIONS

This patent application is a continuation-in-part of U.S. non-provisional patent application Ser. No. 12/430,075, filed Apr. 25, 2009 and U.S. provisional patent application Ser. No. 61/125,977, filed Apr. 30, 2008, each of which is incorporated herein by reference in its entirety.

### FIELD OF THE INVENTION

The present invention is directed to a polymeric pheromone formulation comprising a low melting-point polymer or copolymer, an unsaturated long chain fatty alcohol, and an animal-appeasing pheromone. The present invention is further directed to an animal behavioral control system and method of use wherein the system comprises a polymeric pheromone formulation present in a device that is worn by or placed on a mammal and which allows for the slow-release of the appeasing pheromone. More specifically, the system of the present invention is placed on or in close proximity to an animal to be treated and it releases the appeasing pheromone efficiently and uniformly over an extended period of time of about four weeks or longer.

### BACKGROUND OF THE INVENTION

Stress, by definition, is the reaction of an animal body to forces of deleterious nature, infections and various abnormal states that tend to disturb homeostasis. Animals exposed to stress respond with changes in the activity of the autonomic and neuroendocrine systems and in behavior. The activation of these biological systems is a prerequisite for the animal to cope with stress and thus is the principal resource that will provide the adequate biological defense against a threat that challenges the homeostasis of the animal. Moberg, G. P. Animal Stress, pp. 27-49 (1985); Vogel, W. H. Neuropsychobiology, 13 pp. 1290135 (1985). Mammals are generally are influenced by changes in their environment, which can oftentimes result in them being stressed. Acts such as being taken out of their normal environment, being herded together and transported can often lead to a mammal becoming stressed. As a consequence, pathological disorders, mortality, delays in growth, and behavior disorders often occur in mammals exposed to stressful conditions. Behavior disorders will often lead to aggressive fighting between and/or among animals or can also take the form of being nervous of loud noises, being nervous of people or things in their environment, timid with other animals and/or with people, and dislike of being touched. Mammals, particularly companion animals such as cats and dogs, may additionally exhibit hyperactivity in a home, constant barking or hissing, snapping or biting their owners or others, uncontrolled urination, and the like. This behavior can become so bad that the animal may be given up by its owner and is often euthanized. Stress-related behavior may also be exhibited in various breeds of mammals, for example in livestock such as sheep, horses, pigs, and cattle, by fighting, being highly excitable and agitated, rearing, and trying to escape. For example, piglets begin fighting within hours of birth and when pigs of any age meet, a fight is likely to occur. See, McGlone, J J, Journal of Animal Science, 68:11 pgs. 86-97 (1990).

Pheromone “H,” an artificial animal-appeasing pheromone, or “APH,” has been developed to mimic a calming pheromone secreted by mammals. Current products containing Pheromone “H” are available either in diffuser form or spray bottle form; however, such products are inconvenient as they cannot easily continually accompany an animal and consequently, cannot effectively provide constant modification of an animal's behavior. Accordingly, it is an object of the present invention to develop an APH delivery system and device that can be worn by an animal to be treated and which is always in the presence of an animal in order to provide a consistent and long-term behavior treatment regimen to an animal exhibiting stress-related behavior disorders.

Furthermore, because Pheromone “H” is primarily a blend of natural fatty acids, it is can be difficult to prepare a solid polymeric delivery device for use with the pheromone. The necessity for including high amounts of pheromonal fatty acids in the polymeric article can lead to undesirable results, such as the formation of fatty acid oil residue on the surface of the device, release rates of the pheromone composition that are unacceptably fast, the production of polymeric articles that are too soft and can easily be pulled apart with tension, or polymeric material that cannot be extruded at high or low temperatures. Therefore, it is a further object of the present invention to prepare solid polymeric delivery devices, such as collars, ear tags, or the like, by processing the ingredients at a lower temperature than is normally used in the art of polymeric extrusion.

### SUMMARY OF THE INVENTION

It has now been discovered that by using an unsaturated long chain fatty alcohol together with a low melting-point polymer, a polymeric formulation suitable for the controlled delivery of an animal-appeasing pheromone (i.e. Pheromone “H”) can be developed. More particularly, the present invention is directed to a solid polymeric formulation for controlling behavior in mammals comprising Pheromone “H,” an unsaturated long chain fatty alcohol of from about 11 to about 18 carbon atoms, and a low melting polymer or copolymer. Low melting polymers are polymers and copolymers having a melt temperature of 250° F. or below. The formulation described herein is useful for making articles or devices such as animal collars, tags, solid diffusers, and the like. These articles or devices are also encompassed by the present invention and can be used for releasing the APH to a local environment in contact with the animal, over an extended or prolonged period of time. By “extended or prolonged period of time” is meant for a period of activity longer than the period of activity exhibited by the raw active ingredient alone. The extended period of release can be for at least two weeks, more preferably, at least three weeks, and most preferably at least four weeks.

The present invention is further directed to a method for calming stress-related behavior in mammals, the method comprising placing a polymeric formulation of the present invention, which has been shaped into an appeasing device, in contact with or in the general proximity or presence of the animal to be treated. The polymeric formulation that has been shaped into the appeasing device is capable of remaining with the animal at all times, such that the animal is in the continuous presence of the appeasing pheromone for the useful life of the device; that is, for as long as an effective amount of the APH is released from the device, which can be up to four weeks or longer.

The “fatty alcohol” or “solid fatty alcohol” useful in the present invention is any unsaturated fatty alcohol having from about 6 to about 34 carbon atoms, preferably having from about 11 to about 18 carbon atoms and that is a solid at room temperature. Preferable fatty alcohols include, but are not limited to, cetyl alcohol (which includes: Cetanol, 1-Hexadecanol, Ethal, Ethol, Palmityl alcohol, Hexadecan-1-ol, Hexadecyl alcohol, Hexadecanol, Alcohol C-16, Atalco C, Cachalot C-50, Cetaffine, Cetal, Cetylol, CO-1670, Crodacol-cas, DYTOL F-11, Loroll 24, Loxanol K, and Product 308), stearyl alcohol (which includes: Octadecanol, Stenol, Octadecyl alcohol, Decyl Octyl alcohol, Stearol, N-octadecanol, Adol 68, Alfol 18, Atalco S, CO-1895, CO-1897, Crodacol-S, Dytol E-46, Lorol 28, Polaax, Sipol S, Siponol S, and Steraffine) and Oleyl Alcohol (which includes: Cis-9-Octadecen-1-01). In one aspect, the solid fatty alcohol is present in the formulation of the invention in an amount of from about 5 wt % to about 50 wt % of the formulation. In another aspect, the solid fatty alcohol is present in an amount of from about 12 wt % to about 30 wt % of the total weight of the formulation. In a further aspect, the solid fatty alcohol is present in an amount of from about 12 wt % to about 25 wt % of the formulation.

The “low melting polymer or copolymer” capable of holding and then releasing the pheromone is selected from those polymers or copolymers that are a solid at room temperature and have a melt temperature of below 250° F. In one aspect, the low melting polymer or copolymer has a melt temperature of below 200° F. Examples of polymers and copolymers useful in the present invention include, but are not limited to, polyethylene, polyvinyl acetate, ethylene acid copolymers, ethylene acrylates, polyurethanes, styrene-butadiene, polyvinyl acetate, polyvinyl butyral, and mixtures and copolymers thereof. In one aspect, the low melting polymer or copolymer is present in the formulation of the invention in an amount of from about 40 wt % to about 85 wt %. In another aspect, the low melting polymer or copolymer is present in an amount of from about 65 wt % to about 80 wt % of the total weight of the formulation. In a further aspect, the polymer or copolymer is present in an amount of from about 70 wt % to about 75 wt %.

Appeasing Pheromone “H,” or (APH), is a proprietary composition (from Modern Veterinary Therapeutics, Florida) that comprises linoleic acid, capric acid, pentadecylic acid, azelaic acid, pimelic acid, lauric acid, myristic acid, cholesterol, Cl-docosanol, squalene (shark origin), and BHT. The pheromone is volatile and will decompose at the high temperatures normally used to manufacture pet collars. Additionally, because Pheromone “H” is composed primarily of natural fatty acids, fatty acid oils interfere with the production, physical characteristics, and/or the aesthetics of normal collars. In one aspect, the APH is present in the formulation of the invention in an amount of from about 0.3 wt % to about 20 wt % of the total weight of the formulation. In another aspect of the invention, the APH is present in an amount of from about 3 wt % to about 15 wt %. In a further aspect, the APH is present in an amount of from about 5 wt % to about 10 wt %.

To prepare the polymeric formulations of the present invention, the unsaturated long chain fatty alcohol is added to a low melting polymer or copolymer and mixed until uniformity is achieved. The pheromone is then added with mixing to make a blend. This blend formulation may then be processed into a shaped article or device, such as a pet collar or a tag or the like, on a conventional extruder or molding machine at low temperatures (that is, at a temperature that will melt the low melting polymer or copolymer, which is generally below about 250° F.) by methods known in the art.

Additional components may optionally be included in the polymeric formulation of the present invention. Such optional ingredients can include, but are not limited to, plasticizers, synergists, fragrances, coloring agents, preservatives, antioxidants, light stabilizers, and the like.

After the polymeric formulation is shaped or formed into the desired shape, the article or device, such as a collar, is placed into close proximity with an animal to be treated; that is, the device will be located, by attachment or other means, in sufficient proximity to or contact with the animal such that the APH will, together with the solid fatty alcohol, be released to the surface of the article as a result of the animal's body heat. The combination of the unsaturated long chain fatty alcohol and the low-melting polymer allows the pheromone to be released to the surface of the polymeric formulation continuously over a period of at least four weeks while it is simultaneously being transferred to the animal's hair and down to the skin. One skilled in the art will also recognize that the article or device may take other shapes suitable for use with animals in which a collar would be difficult to apply, for example, rodents such as mice and rats. Therefore, for particular animals, the device may take the shape of, for example, a solid strip that can be hung on a wall or from a rafter or ceiling whereby the strip is placed in close proximity to the animal.

The appeasing formulation of the present invention can be obtained and used with any mammal, for example, in dogs, cats, horses, or ferrets to calm their anxiety after, for example, removal from their familiar surroundings such as being taken to the veterinarian.

### DEFINITIONS

As used herein, the word “mammal” is interchangeable with the word “animal” and encompasses any group of vertebrates the females of which have milk-secreting glands, including man. Examples of mammals include, but are not limited to, cats, dogs, horses, humans, cattle, pigs, rats, rabbits, apes, chimpanzees and the like.

As used herein, the word “stress” refers to the reaction of an animal body to forces of deleterious nature, infections and various abnormal states that tend to disturb homeostasis. This reaction may be a physical reaction or an emotional reaction including anxiety.

As used herein, the word “pheromone” refers to a substance released by the body of a particular species that causes a predictable reaction by another individual of the same species, which substance may serve, for example, as a specific attractant, social communicator, sexual stimulant and the like.

The following examples illustrate the practice of the present invention, but are not limiting. Parts are given as percentages and temperatures are in degrees Fahrenheit unless otherwise noted.

### EXAMPLE 1

The formulation in Table 1 (below) is prepared, and is then formed into an animal collar, as follows:

TABLE 1

| | Percentage |
|---|---|
| | of Total |
| Ingredients: | Formulation: |
| Pheromone “H” | 6.0 |
| Cetyl Alcohol | 20.0 |
| Polymer MU- | 73.8 |
| 76000 | |
| Lime Green | 0.2 |
| colorant | |

### Sources:

Pheromone “H”—proprietary blend, Modern Veterinary Therapeutics.
Cetyl alcohol (1-hexadecanol)—Procter & Gamble.
Microthene® Polymer MU-76000 (ethylene-vinyl acetate copolymer, vinyl acetate content: 18 wt %, ground powder, melt index: 32 g/10 min (EMI), avg. particle size: 35 mesh)—Equistar Chemicals, LP.

### Colorant—Day Glow Color Corp.

### Mixing Procedure:

1. The polymer is weighed and placed into a mixing vessel.
2. The colorant is added to the polymer with mixing.
3. The cetyl alcohol is added to the polymer and mixed until uniform.
4. Optional Step: Fragrance is added to the Pheromone “H” active phase and mixed into the DAPH before adding to the polymer/fatty alcohol mixture. (This step is optional if fragrance is desired).
5. The Pheromone “H” (with optional fragrance) is added to the polymer/fatty alcohol mixture while mixing, and mixing is continued for 1-2 hours.
6. The resulting blend is allowed to cool to room temperature, preferably overnight to allow for easy feeding of the blend while extruding.
The blend is then extruded or molded, with temperature settings at 190° F. for zones 1, 2, 3 and 4, into the shape desired, which in this Example was an animal collar. The animal collar can then be used on any animal described in the present invention including companion animals or livestock. The formulation and size of the collar can be adjusted dependent on the size of the animal it was created for use.

### EXAMPLES 2-6

Following the procedures of Example 1, animal collars were prepared from each of the formulations in Table 2, Table 3, Table 4, Table 5, and Table 6, below.

TABLE 2

| | Percentage of |
|---|---|
| | total weight |
| | of the |
| Ingredients: | Formulation: |
| Pheromone “H” | 6.0 |
| Cetyl Alcohol | 20.0 |
| Polymer MU- | 70.8 |
| 76000 | |
| Lime Green | 0.2 |
| colorant | |
| Fragrance | 3.0 |

TABLE 3

| | Percentage of |
|---|---|
| | total weight of |
| Ingredients: | the formulation: |
| Pheromone “H” | 6.0 |
| Cetyl Alcohol | 12.5 |
| Polymer MU- | 78.3 |
| 76000 | |
| Lime Green | 0.2 |
| colorant | |
| Fragrance | 3.0 |

TABLE 4

| | Percentage of |
|---|---|
| | total weight of |
| Ingredients: | the formulation: |
| Pheromone “H” | 9.0 |
| Cetyl Alcohol | 20.0 |
| Polymer MU- | 70.8 |
| 76000 | |
| Lime Green | 0.2 |
| colorant | |

TABLE 5

| | Percentage of |
|---|---|
| | total weight of |
| Ingredients: | the formulation: |
| Pheromone “H” | 9.0 |
| Cetyl Alcohol | 20.0 |
| Polymer MU- | 67.8 |
| 76000 | |
| Lime Green | 0.2 |
| colorant | |
| Fragrance | 3.0 |

TABLE 6

| | Percentage of |
|---|---|
| | total weight of |
| | the |
| Ingredients: | formulation: |
| Pheromone “H” | 9.0 |
| Cetyl Alcohol | 12.5 |
| Polymer MU- | 75.3 |
| 76000 | |
| Lime Green | 0.2 |
| colorant | |
| Fragrance | 3.0 |

### EXAMPLE 7

The collars of Example 1 were subjected to efficacy evaluation. The collars were placed onto dogs that had stress-related behavioral problems, such as being nervous of noises (trembling), timid with other dogs and people, hyperactive in the house, barking at everything, shielding away from other dogs, overly playful, chew a lot, destructive inside the house, very snappy with owners, do not like to be fussed with or touched, and/or constantly barking at owners for attention. The dogs remained in their home environments for the period of the test.

After the APH collars were applied to the dogs, 80 wt % of the treated dogs experienced improvements: some dogs were more relaxed, trembling stopped, confidence was restored, hyperactivity stopped, great improvement shown when dealing with others, dogs appeared happier, and excessive barking stopped.

At the end of four weeks the collars were removed from the dogs, after which a majority of the dogs reverted back to being nervous (trembling) and timid, were not so confident, were hyperactive, and started to bark a lot.

During the 3-month testing period, visible evidence was observed as to the functionality of the DAPH collars. The majority of the testing subjects exhibited positive results after using the collar and most regressed to their previous behaviors once its use was suspended. Most subjects' owners expressed a desire to purchase the collars.

### EXAMPLE 8

The collars of Example 1 and their efficacy on cats was tested and assessed. Cat owners were recruited at random from a database of known cat owners. The cats qualified for the study if they exhibited one or more of the problem behaviors (vertical scratching, urine marking, hissing, hiding, aggression towards people and/or other animals, and excessive meowing) at least twice weekly with a nuisance factor (for the owners) of at least 6 on a 10-point scale. Cats were at least six months old. Age, weight, and short hair vs. long hair were recorded, but were not screening factors.

There were three collar cells of approximately 34 each (several cats had multiple problems), and one cell of 36 diffuser users that served as a control. The collars were provided to the owners and remained on the cats for a period of thirty days.

Among collar users with 2 or 3 cats, 44 wt % noticed a calming effect, vs. 29 wt % of single cat households. In addition, 35 wt % of multi-cat owners noticed better behavior with dogs or other cats and 26 wt % reported reduced or eliminated aggression, vs. 13 wt % and 7 wt %, respectively, of single cat households. Among all of the subjects, over 20 wt % rated the collars as “excellent”, compared to only 3 wt % of diffuser users and 68 wt % of the collar users reported positive comments about behavioral efficacy, compared to 44 wt % of the diffuser users.

### EXAMPLE 9

The collars of Example 1 and their efficacy on horses was tested and assessed. It was observed that when the collar was placed in close proximity to a horse, the horse appeared to be more relaxed than before being subjected to the pheromone collar.

While the invention has been explained in relation to exemplary embodiments, it is to be understood that many variations, modifications, and changes to the process described herein are possible and will become apparent to those skilled in the art upon reading the description. Therefore, it is to be understood the invention disclosed herein is intended to cover such variations, modifications, and changes which do not depart from the spirit and scope of the present invention, which invention is limited only by the claims which follow.

1. A polymeric formulation for the controlled delivery of an animal-appeasing pheromone, the polymeric formulation comprising a low melting polymer or copolymer, an unsaturated or saturated long-chain fatty alcohol having from about 6 to about 34 carbon atoms, and an animal-appeasing pheromone.

2. The polymeric formulation according to claim 1, wherein the animal-appeasing pheromone is Pheromone “H,” which is present in the polymeric formulation in an amount between about 0.3 wt % to about 20 wt % of the total weight of the formulation.

3. The polymeric formulation according to claim 1, wherein the low melting polymer or copolymer is selected from the group consisting of polyethylene, polyvinyl acetate, ethylene acid copolymers, ethylene acrylates, polyurethanes, styrene-butadiene, polyvinyl acetate, polyvinyl butyral, and combinations thereof, and wherein the low melting polymer or copolymer is present in the polymeric formulation in an amount between about 40 wt % to about 85 wt % of the total weight of the formulation.

4. The polymeric formulation according to claim 1, wherein the long-chain fatty alcohol is selected from the group consisting of cetyl alcohol, stearyl alcohol, and oleyl alcohol, and wherein the long-chain fatty alcohol is present in the polymeric formulation in an amount from about 5 wt % to about 50 wt % of the total weight of the formulation.

5. The polymeric formulation according to claim 1, wherein the polymeric formulation is shaped into a collar.

6. The polymeric formulation according to claim 5, wherein the collar is suitable for a mammal.

7. The polymeric formulation according to claim 6, wherein the mammal is selected from the group consisting of cats, dogs, horses, cattle, pigs, rats, rabbits, apes, and chimpanzees.

8. A polymeric formulation for controlling behavior in mammals, the formulation comprising a low melting polymer or copolymer, an unsaturated or saturated long-chain fatty alcohol of from about 6 to about 34 carbon atoms, and an animal-appeasing pheromone.

9. The polymeric formulation according to claim 8, wherein the animal-appeasing pheromone is Pheromone “H,” which is present in the polymeric formulation in an amount from about 5 wt % to about 10 wt % total weight of the formulation.

10. The polymeric formulation according to claim 8, wherein the low melting polymer or copolymer is selected from the group consisting of polyethylene, polyvinyl acetate, ethylene acid copolymers, ethylene acrylates, polyurethanes, styrene-butadiene, polyvinyl acetate, polyvinyl butyral, and combinations thereof, and wherein the low melting polymer or copolymer is present in the polymeric formulation in an amount from about 40 wt % to about 85 wt % of the total weight of the formulation.

11. The polymeric formulation according to claim 8, wherein the long-chain fatty alcohol is selected from the group consisting of cetyl alcohol, stearyl alcohol, and oleyl alcohol, and wherein the long-chain fatty alcohol is present in the polymeric formulation in an amount from about 12 wt % to about 30 wt % of the total weight of the formulation.

12. The polymeric formulation according to claim 8, wherein the polymeric formulation is shaped into a collar.

13. The polymeric formulation according to claim 12, wherein the collar is suitable for a mammal.

14. The polymeric formulation according to claim 13, wherein the mammal is selected from the group consisting of cats, dogs, horses, cattle, pigs, rats, rabbits, apes, and chimpanzees.

15. A method for calming stress-related behavior in mammals, the method comprising the step of:
placing a polymeric formulation for controlling behavior in mammals in close proximity with a mammal to be treated, wherein the polymeric formulation comprises a low melting polymer or copolymer, an unsaturated or saturated long-chain fatty alcohol of from about 6 to about 34 carbon atoms, and an animal-appeasing pheromone.

16. The method according to claim 15, wherein the animal-appeasing pheromone is Pheromone “H.”

17. The method according to claim 15, wherein the polymeric formulation is shaped into a collar.

18. The method according to claim 17, wherein the collar is suitable for a mammal.

19. The method according to claim 18, wherein the mammal is selected from the group consisting of cats, dogs, horses, cattle, pigs, rats, rabbits, apes, and chimpanzees.

20. A method for preparing a polymeric formulation for the control of animal
behavior, the method comprising the steps of:
a. mixing a low melting polymer or copolymer with a long-chain fatty alcohol;
b. adding an animal-appeasing pheromone to the low melting polymer and fatty alcohol to form a polymeric blend;
c. processing the polymeric blend at a temperature at or below 250° F.; and
d. forming the polymeric blend into a shaped article or device.

21. The method according to claim 20, wherein the animal-appeasing pheromone is Pheromone “H.”

22. The method according to claim 20, wherein the shaped article or device is a collar.

23. The method according to claim 22, wherein the collar is suitable for a mammal.

24. The method according to claim 23, wherein the mammal is selected from the group consisting of cats, dogs, horses, cattle, pigs, rats, rabbits, apes, and chimpanzees.
